(19) United States
(12) Patent Application Publication
(10) Pub. No.: US 2023/0380675 A1
(43) Pub. Date: Nov. 30, 2023

(54) ENDOSCOPE SYSTEM

(71) Applicant: Panasonic Intellectual Property Management Co., Ltd., Osaka (JP)

(72) Inventors: Shogo MOTEGI, Osaka (JP); Shintaro HAYASHI, Hyogo (JP); Fumiya YAGI, Osaka (JP); Toshiaki TAKENAKA, Osaka (JP); Takeshi ABE, Osaka (JP); Natsuki KITO, Kyoto (JP)

(73) Assignee: Panasonic Intellectual Property Management Co., Ltd., Osaka (JP)

(21) Appl. No.: 18/031,497

(22) PCT Filed: Oct. 5, 2021
(86) PCT No.: PCT/JP2021/036855

§ 371 Date: Apr. 12, 2023

(30) Foreign Application Priority Data
Oct. 20, 2020 (JP) 2020-176100

(51) Int. Cl. A61B 1/06; A61B 1/07; A61B 1/00

(52) U.S. Cl. CPC A61B 1/0638; A61B 1/07; A61B 1/0669; A61B 1/0655; A61B 1/0646; A61B 1/00097; A61B 1/063

(57) ABSTRACT

An endoscope system includes: an insertion component which includes a base end and a tip end, and is inserted into a subject at least up to the tip end; a light source which emits first light and second light having a hue different from a hue of the first light a light sensor; and a controller, the insertion component includes: a first light guide member which emits the first light and the second light from the tip end as illumination light; and a second light guide member which guides reflected light of the illumination light emitted from the tip end, the light sensor receives the reflected light guided by the second light guide member, and the controller controls the light source based on the intensity of the reflected light received by the light sensor to stop emission of the at least one of the first light or the second light.

### TECHNICAL FIELD

The present invention relates to endoscope systems.

### BACKGROUND ART

Conventionally, an endoscope system is known which includes an insertion component that is inserted into a subject in order to observe the interior of the subject. For example, an endoscope system disclosed in Patent Literature (PTL) 1 includes a laser probe which guides laser light into the insertion component, an illuminator, and an imager. The imager receives light which is emitted from the illuminator and is then reflected inside the subject and light which is emitted from a tip end of the laser probe and is then reflected inside the subject, and thereby allows the observation of the interior of the subject.

### CITATION LIST

### Patent Literature

[PTL 1] Japanese Patent No. 5792415

### SUMMARY OF INVENTION

### Technical Problem

In an endoscope system, the diameter of an insertion component is required to be reduced so that a burden on a subject is reduced.

Hence, an object of the present invention is to provide an endoscope system which can reduce the diameter of an insertion component.

### Solution to Problem

An endoscope system according to an aspect of the present invention includes: an insertion component which includes a base end and a tip end, and is inserted into a subject at least up to the tip end; a light source which emits first light and second light having a hue different from a hue of the first light; a light sensor which is sensitive to a wavelength of at least one of the first light or the second light; and a controller which controls the light source based on the result of detection performed by the light sensor, the insertion component includes: a first light guide member which guides, from the base end to the tip end, the first light and the second light emitted from the light source so that the first light and the second light are emitted from the tip end as illumination light; and a second light guide member which guides, from the tip end to the base end, reflected light of the illumination light emitted from the tip end, the light sensor receives the reflected light guided by the second light guide member, and the controller controls the light source based on the intensity of the reflected light received by the light sensor to stop emission of at least one of the first light or the second light.

### Advantageous Effects of Invention

In an endoscope system according to the present invention, it is possible to reduce the diameter of an insertion component.

### BRIEF DESCRIPTION OF DRAWINGS

FIG. 1 is a diagram showing the configuration of an endoscope system according to an embodiment and a path of light when an insertion component is inserted into a subject.

FIG. 2 is a diagram showing the configuration of the endoscope system according to the embodiment and a path of light when the insertion component is not inserted into the subject.

FIG. 3 is a flowchart showing the operation of the endoscope system according to the embodiment.

FIG. 4A is a diagram showing the configuration of a light sensor according to variation 1.

FIG. 4B is a diagram showing the configuration of a light sensor according to variation 2.

### DESCRIPTION OF EMBODIMENTS

An endoscope system according to an embodiment of the present invention will be described in detail below with reference to drawings. The embodiment described below shows a specific example of the present invention. Hence, numerical values, shapes, materials, constituent elements, the arrangement and connection of the constituent elements, steps, the order of the steps, and the like shown in the following embodiment are examples, and are not intended to limit the present invention. Therefore, among the constituent elements in the following embodiment, constituent elements which are not recited in the independent claim are described as optional constituent elements.

The drawings are schematic views and are not exactly shown. Hence, for example, scales and the like are not necessarily the same in the drawings. In the drawings, substantially the same configurations are identified with the same reference signs, and repeated descriptions are omitted or simplified.

In the present specification, terms such as parallel which indicate relationships between elements, terms which indicate the shapes of elements, and numerical ranges are expressions which not only indicate exact meanings but also indicate substantially equivalent ranges such as a range including a several percent difference.

In the present specification, a “peak wavelength” is a wavelength at which the intensity of light emission is the maximum within a predetermined wavelength band. The intensity at the peak wavelength is referred to as a “peak intensity”. The peak intensity does not need to be the maximum intensity in all wavelength bands. In other words, a peak whose intensity is higher than the peak intensity within the predetermined wavelength band may be present in a band other than the predetermined wavelength band.

In the present specification, unless otherwise specified, ordinal numbers such as “first” and “second” do not mean the number or order of constituent elements but are used to avoid confusion of similar constituent elements and to distinguish between them.

### EMBODIMENT

### [Configuration]

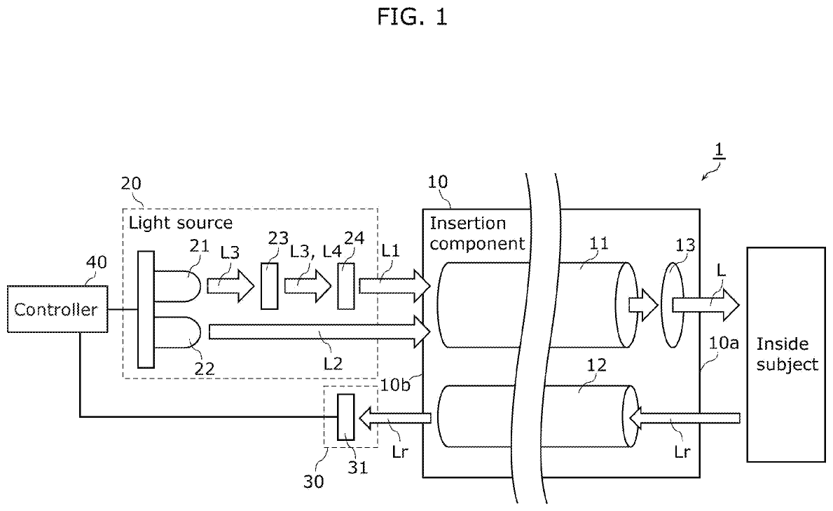

The configuration of the endoscope system according to the present embodiment will first be described with reference to FIG. 1.

FIG. 1 is a diagram showing the configuration of endoscope system 1 according to the present embodiment. Endoscope system 1 shown in FIG. 1 is used to observe the interior of a subject. The subject is, for example, a living body such as a human or animal body. Endoscope system 1 is utilized for at least one of a normal observation or a special observation.

The normal observation is to utilize white light to obtain a visible light image inside the subject. The special observation is, for example, an observation called narrow band imaging (NBI) in which narrow-band light is utilized. In the NBI, two types of narrow-band light having different wavelengths are utilized, and thus an image in which a specific object such as a blood vessel is emphasized is obtained.

As shown in FIG. 1, endoscope system 1 includes insertion component 10, light source 20, light sensor 30, and controller 40.

Insertion component 10 includes tip end 10a and base end 10b, and at least tip end 10a is inserted into the subject. Light source 20 and light sensor 30 are connected to base end 10b. Light source 20 and light sensor 30 are not inserted into the subject.

Light source 20 emits first light L1 and second light L2. Second light L2 is light which has a hue different from a hue of first light L1. Specifically, the peak wavelength of second light L2 is different from the peak wavelength of first light L1. For example, first light L1 is green light, and second light L2 is violet light.

Each of green light and violet light is light in a wavelength band which is absorbed by hemoglobin included in blood vessels. Hence, when first light L1 (green light) and second light L2 (violet light) are applied into the subject, each of first light L1 and second light L2 is absorbed by capillaries in a mucosal surface layer and large blood vessels in a deep portion. Electrical signals corresponding to the intensities of the green light and the violet light are processed, and thus it is possible to obtain an image in which blood vessels are enhanced.

Light sensor 30 is sensitive to a wavelength of at least one of first light L1 or second light L2. In the present embodiment, light sensor 30 includes sensor 31. Sensor 31 receives reflected light Lr of illumination light L emitted from tip end 10a of insertion component 10, and generates and outputs an electrical signal corresponding to the intensity of the received light. Sensor 31 is, for example, a photoelectric conversion element such as a photodiode or phototransistor.

Controller 40 controls light source 20. Specifically, controller 40 controls light source 20 based on the intensity of reflected light Lr received by light sensor 30 to stop the emission of the at least one of first light L1 or second light L2. The specific operation of controller 40 will be described later.

Controller 40 is realized, for example, by a large scale integration (LSI) circuit which is an integrated circuit (IC). The integrated circuit is not limited to the LSI circuit, and may be a dedicated circuit or a general-purpose processor. For example, controller 40 may be a microcontroller. The microcontroller includes, for example, a non-volatile memory in which a program is stored, a volatile memory which is a temporary storage area for executing the program, input/output ports, a processor which executes the program, and the like. Controller 40 may be a field programmable gate array (FPGA) which can be programmed or a reconfigurable processor in which the connection and settings of circuit cells in an LSI circuit can be reconfigured. Functions which are performed by controller 40 may be realized by software or hardware.

The specific configurations of the constituent elements of endoscope system 1 will be described below with reference to FIG. 1. The specific configuration of insertion component 10 will first be described.

As shown in FIG. 1, insertion component 10 includes first optical fiber 11, second optical fiber 12, and objective lens 13. Insertion component 10 includes a cylindrical member (not shown) which houses and holds first optical fiber 11 and second optical fiber 12 thereinside. The cylindrical member includes a first channel for inserting first optical fiber 11 and a second channel for inserting second optical fiber 12. The first channel and the second channel each are through holes which penetrate the cylindrical member in an axial direction and are separated from each other. For example, the cylindrical member is formed of a light-shielding material. In this way, it is possible to suppress light interference and the like based on light leakage between the two optical fibers.

First optical fiber 11 is an example of a first light guide member, and guides first light L1 and second light L2 emitted from light source 20 from base end 10b to tip end 10a and outputs first light L1 and second light L2 from tip end 10a as illumination light L. Since objective lens 13 is arranged at the end of first optical fiber 11 on the side of tip end 10a, illumination light L is emitted via objective lens 13.

Insertion component 10 may include, instead of first optical fiber 11, an optical fiber for guiding first light L1 and an optical fiber for guiding second light L2. In this case, insertion component 10 may include channels through which the two optical fibers are individually inserted.

Second optical fiber 12 is an example of a second light guide member, and guides reflected light Lr of illumination light L emitted from tip end 10a from tip end 10a to base end 10b. Although not shown in FIG. 1, a condenser lens may be provided at the end of second optical fiber 12 on the side of tip end 10a. The condenser lens focuses light from outside insertion component 10 onto the end of second optical fiber 12.

The specific configuration of light source 20 will then be described.

As shown in FIG. 1, light source 20 includes first light source 21, second light source 22, wavelength converter 23, and filter 24.

First light source 21 emits third light L3. Third light L3 is light which has a different wavelength from first light L1 and second light L2. First light source 21 is a laser light source. In the present embodiment, third light L3 is blue light which has a peak wavelength in a wavelength band of 430 nm or more and 495 nm or less. Third light L3 is narrow-band light. Specifically, although the half width of third light L3 is less than or equal to 20 nm, the half width may be less than or equal to 10 nm or may be less than or equal to 5 nm. For example, first light source 21 is a blue laser light source which has a peak wavelength of 455 nm.

Second light source 22 emits second light L2. Second light source 22 is a laser light source. In the present embodiment, second light L2 has a wavelength shorter than the peak wavelength of third light L3. Specifically, second light L2 is violet light which has a peak wavelength in a wavelength band that is greater than or equal to 380 nm and less than 430 nm. Second light L2 is narrow-band light. Specifically, although the half width of second light L2 is less than or equal to 20 nm, the half width may be less than or equal to 10 nm or may be less than or equal to 5 nm. For example, second light source 22 is a violet laser light source which has a peak wavelength of 415 nm.

In the present embodiment, first light source 21 and second light source 22 are arranged side by side such that their optical axes are parallel to each other. The optical axis of the light source is the center of the light emitted from the light source, and coincides with the direction of emission of the light. For example, first light source 21 and second light source 22 are provided on the same substrate.

Wavelength converter 23 includes a plurality of fluorescent substances. For example, each of the fluorescent substances is a particulate material. Wavelength converter 23 includes, for example, a plate-shaped resin base material (not shown), and in the resin base material, the fluorescent substances are dispersed. Alternatively, wavelength converter 23 may include a transparent plate material, and the fluorescent substances may be dispersed in a transparent resin applied on the surface of the plate material. Wavelength converter 23 may be an aggregate (for example, a ceramic sintered material) in which a plurality of fluorescent substances are aggregated.

The fluorescent substances each absorb a part of third light L3 and emit fourth light L4 which has a hue different from a hue of third light L3. In other words, third light L3 functions as excitation light for the fluorescent substances. In the present embodiment, the peak wavelength of fourth light L4 is longer than the peak wavelength of third light L3. Specifically, fourth light L4 is yellow light which has a peak wavelength in a wavelength band of 525 nm or more and 600 nm or less. Fourth light L4 has an intensity greater than or equal to a predetermined percentage of the peak intensity over the wavelength band of 500 nm or more and 600 nm or less. Although the predetermined percentage is, for example, 5%, the predetermined percentage may be 10% or may be 20%. In other words, fourth light L4 is broad light which has a larger half width than third light L3. For example, the fluorescent substance is a Y₃Al₅O₁₂:Ce³⁺ fluorescent substance (YAG:Ce³⁺) which has an excitation wavelength of 455 nm and a peak wavelength of 545 nm. As long as the fluorescent substance has a fluorescence spectrum equivalent to YAG:Ce³⁺, instead of or in addition to YAG:Ce³⁺, another yellow fluorescent substance may be used.

Wavelength converter 23 is arranged on the optical path of third light L3. When third light L3 enters wavelength converter 23, wavelength converter 23 emits combined light of another part of third light L3 (that is, light which is not absorbed by the fluorescent substances) and fourth light L4. At least a part of the combined light is first light L1.

Filter 24 is a filter which transmits light of a specific wavelength included in the combined light emitted from wavelength converter 23. In the present embodiment, light which has transmitted filter 24 is first light L1.

Filter 24 is a bandpass filter which has a passband in a wavelength band which is greater than or equal to 505 nm and less than 525 nm. Although the width of the passband is less than 20 nm, the width may be less than or equal to 10 nm or may be less than or equal to 5 nm. For example, first light L1 which has passed through filter 24 is green light which has a peak wavelength of 515 nm.

The specific configuration of light sensor 30 will then be described.

As described above, light sensor 30 includes sensor 31. Sensor 31 is sensitive to a wavelength of at least one of first light L1 or second light L2. For example, sensor 31 is sensitive to each of first light L1 and second light L2. As an example, sensor 31 is sensitive to a visible light band (range of 380 nm or more and 780 nm or less). Sensor 31 photoelectrically converts reflected light Lr to generate an electrical signal indicating the intensity of reflected light Lr. The generated electrical signal is output to controller 40.

In the present embodiment, light sensor 30 is a dedicated light sensor which is used for determining whether to stop light source 20. Endoscope system 1 separately includes a sensor for obtaining an image inside the subject.

For example, although it is not shown in FIG. 1, endoscope system 1 further includes an image sensor which generates an observation image inside the subject. Although the image sensor includes a plurality of pixels aligned two-dimensionally, and each of the pixels includes, for example, sub-pixels corresponding to R (red), G (green), and B (blue), the image sensor is not limited to this configuration. Although the image sensor is arranged in the vicinity of, for example, tip end 10a of insertion component 10, the image sensor is not limited to this configuration. As with light source 20, light sensor 30, and controller 40, the image sensor may be provided in a part other than insertion component 10, that is, a part which is not inserted into the subject.

### [Operation]

The operation of endoscope system 1 according to the present embodiment will then be described.

Since illumination light L includes violet light having a short wavelength, it is not desirable that illumination light L directly enter the human eye for a long period of time. When tip end 10a of insertion component 10 is inserted into the subject, illumination light L does not substantially leak out, and thus illumination light L does not enter the human eye. On the other hand, when tip end 10a of insertion component 10 is removed from inside the subject, illumination light L may enter the human eye. Hence, it is expected that the emission of illumination light L is rapidly stopped.

In endoscope system 1 according to the present embodiment, controller 40 controls light source 20 based on the intensity of reflected light Lr of illumination light L. Specifically, controller 40 utilizes a difference between the intensity of reflected light Lr inside the subject and the intensity of reflected light Lr outside the subject, and thus when tip end 10a of insertion component 10 is removed to the outside, controller 40 stops the emission of illumination light L.

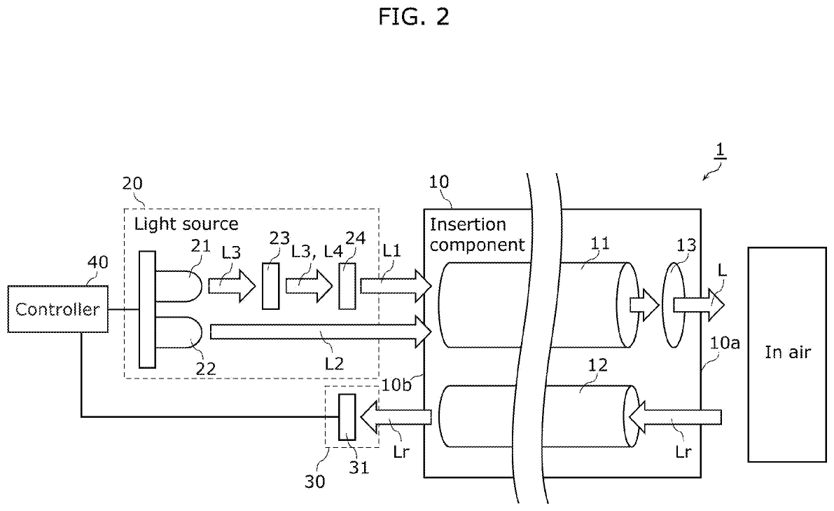

FIG. 2 is a diagram showing the configuration of endoscope system 1 according to the present embodiment and a path of light when insertion component 10 is not inserted into the subject. As is seen by comparison of FIGS. 1 and 2, when tip end 10a of insertion component 10 is located outside the subject (FIG. 2), the intensity of reflected light Lr is increased as compared with a case where tip end 10a is located inside the subject (FIG. 1). This is for the following reason.

When as shown in FIG. 1, tip end 10a of insertion component 10 is located inside the subject, both first light L1 (green light) and second light L2 (violet light) are absorbed by hemoglobin in blood. Hence, the intensity of reflected light Lr is decreased. On the other hand, when as shown in FIG. 2, tip end 10a of insertion component 10 is located outside the subject, first light L1 (green light) and second light L2 (violet light) are not absorbed by hemoglobin. Hence, the intensity of reflected light Lr is increased. In each of the figures, the difference in intensity is schematically represented by the thickness of white arrows.

Thus, when the intensity of reflected light Lr is low, tip end 10a of insertion component 10 can be regarded as being located inside the subject. When the intensity of reflected light Lr is high, tip end 10a of insertion component 10 can be regarded as being located outside the subject. Therefore, controller 40 can determine, based on the intensity of reflected light Lr, whether tip end 10a of insertion component 10 is located inside or outside the subject, and when tip end 10a is located outside the subject, controller 40 can stop the emission of the light from light source 20.

Specifically, controller 40 compares, with a threshold value, a relative value of the intensity of reflected light Lr of a predetermined wavelength to a reference value. The reference value is the intensity of reflected light Lr of the predetermined wavelength when tip end 10a is located inside the subject as shown in FIG. 1. The predetermined wavelength is, for example, the peak wavelength of second light L2. In other words, the reference value is the intensity of the reflected light of the violet light (second light L2) adsorbed by hemoglobin. The reference value can be determined based on the electrical signal output from sensor 31 when tip end 10a is located inside the subject. The threshold value is determined as a value capable of distinguishing between a case where tip end 10a is located inside the subject and a case where tip end 10a is not located inside the subject.

Controller 40 stops the emission of the at least one of first light L1 or second light L2 when the relative value is higher than the threshold value. For example, controller 40 stops the emission of both first light L1 and second light L2. In this way, it is possible to suppress the emission of strong light when tip end 10a of insertion component 10 is located outside the subject.

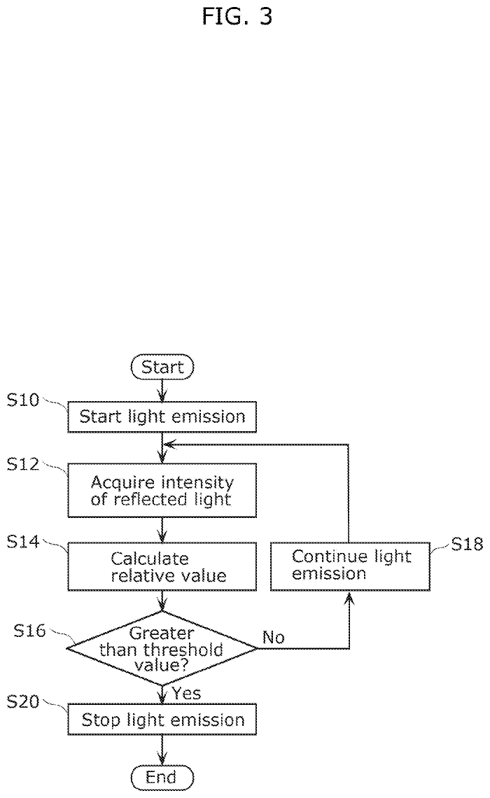

FIG. 3 is a flowchart showing the operation of endoscope system 1 according to the present embodiment.

As shown in FIG. 3, controller 40 first starts light emission which is performed by light source 20 (S10). For example, controller 40 starts the light emission performed by light source 20 when acquiring an operation input for indicating the start of the light emission. For example, endoscope system 1 includes an operation button (not shown) or the like.

In this way, light source 20 emits first light L1 and second light L2. First light L1 and second light L2 emitted are guided through first optical fiber 11 and are emitted from tip end 10a as illumination light L. Illumination light L emitted from tip end 10a is reflected inside or outside the subject, and reflected light Lr thereof is guided through second optical fiber 12 from tip end 10a to base end 10b. Reflected light Lr emitted from base end 10b is received by light sensor 30, and thus an electrical signal corresponding to the intensity of reflected light Lr is generated.

Then, controller 40 acquires the intensity of reflected light Lr (S12). Specifically, controller 40 acquires the intensity of reflected light Lr of a predetermined wavelength based on the electrical signal generated by light sensor 30. For example, controller 40 acquires the intensity of violet light included in reflected light Lr.

Then, controller 40 calculates the relative value of the acquired intensity to the reference value (S14), and compares the calculated relative value with the threshold value (S16). The reference value and the threshold value are previously determined and are stored in a memory or the like included in controller 40.

When the relative value is less than or equal to the threshold value (no in S16), controller 40 continues the light emission performed by light source 20 (S18). The case where the relative value is less than or equal to the threshold value corresponds to the fact that the violet light is absorbed by hemoglobin included in blood of the subject, that is, a case where tip end 10a of insertion component 10 is inserted into the subject. Hence, even when the light emission is continued, the light does not enter the human eye.

When the relative value is greater than the threshold value (yes in S16), controller 40 stops the light emission performed by light source 20 (S20). The case where the relative value is greater than the threshold value corresponds to the fact that the violet light is not absorbed by hemoglobin included in blood of the subject, that is, a case where tip end 10a of insertion component 10 is not inserted into the subject so as to be located outside the subject. Hence, the light emission is stopped, and thus it is possible to suppress the application of the strong light to the human eye.

As described above, in endoscope system 1 according to the present embodiment, when tip end 10a of insertion component 10 is located outside the subject and an operator erroneously causes light source 20 to emit light or when tip end 10a of insertion component 10 inserted into the subject is removed from inside the subject before the light emission performed by light source 20 is stopped, it is possible to rapidly stop the light emission.

Since in the present embodiment, light sensor 30 receives the light of the same wavelength as the light emitted by light source 20, controller 40 can accurately determine, while suppressing the influence of disturbances, whether tip end 10a of insertion component 10 is located inside or outside the subject. Hence, controller 40 can accurately determine whether the light emission performed by light source 20 is stopped.

Although the example where the intensity of the violet light (second light L2) is utilized is described here as an example, the present invention is not limited to this example. Instead of the violet light, the intensity of the green light (first light L1) may be utilized. Alternatively, the intensity of combined light of the violet light and the green light may be utilized.

### [Variations]

The configuration of light sensor 30 is not limited to the example shown in FIG. 1. For example, light sensor 30 may include a plurality of sensors which are respectively sensitive to different wavelength bands.

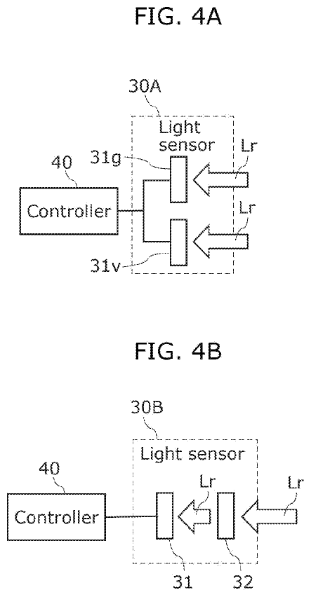

FIG. 4A is a diagram showing the configuration of light sensor 30A according to variation 1 of the embodiment. As shown in FIG. 4A, light sensor 30A includes first sensor 31g and second sensor 31v. The wavelength bands to which first sensor 31g and second sensor 31v are sensitive are narrow bands, and the narrow bands do not overlap each other.

First sensor 31g is a sensor which is sensitive to the peak wavelength of first light L1. Specifically, first sensor 31g is sensitive to green light but is not sensitive to the light other than green light. For example, first sensor 31g is sensitive to a wavelength band which is greater than or equal to 505 nm and less than 525 nm but is not sensitive to wavelength bands other than the wavelength band.

In this way, first sensor 31g can generate an electrical signal corresponding to the intensity of the component of the same wavelength as first light L1 which is included in reflected light Lr. Specifically, first sensor 31g photoelectrically converts the green light included in reflected light Lr to generate the electrical signal indicating the intensity of the green light.

Second sensor 31v is a sensor which is sensitive to the peak wavelength of second light L2. Specifically, second sensor 31v is sensitive to green light but is not sensitive to the light other than violet light. For example, second sensor 31v is sensitive to a wavelength band which is greater than or equal to 380 nm and less than 430 nm but is not sensitive to wavelength bands other than the wavelength band.

In this way, second sensor 31v can generate an electrical signal corresponding to the intensity of the component of the same wavelength as second light L2 which is included in reflected light Lr. Specifically, second sensor 31v photoelectrically converts the violet light included in reflected light Lr to generate the electrical signal indicating the intensity of the violet light. The electrical signals generated in first sensor 31g and second sensor 31v are output to controller 40.

FIG. 4B is a diagram showing the configuration of light sensor 30B according to variation 2 of the embodiment. As shown in FIG. 4B, light sensor 30B includes sensor 31 and filter 32. Sensor 31 is the same as sensor 31 shown in FIG. 1.

Filter 32 is a filter which transmits the light of a predetermined wavelength. The light which has passed through filter 32 enters sensor 31.

Filter 32 has a passband which includes a peak wavelength of at least one of first light L1 or second light L2. For example, filter 32 is a bandpass filter which has a passband in a wavelength band (green light) that includes the peak wavelength of first light L1 and is greater than or equal to 505 nm and less than 525 nm. Filter 32 may also be a bandpass filter which has a passband in a wavelength band (violet light) that includes the peak wavelength of second light L2 and is greater than or equal to 380 nm and less than 430 nm. Filter 32 may also be a multi-bandpass filter which has passbands in a plurality of wavelength bands (for example, green light and violet light). Although the width of the passband is less than 20 nm, the width may be less than or equal to 10 nm or may be less than or equal to 5 nm.

The light of the predetermined wavelength which has passed through filter 32 enters sensor 31. Hence, for example, sensor 31 can generate an electrical signal indicating the intensity of the green light included in reflected light Lr or an electrical signal indicating the intensity of the violet light included in reflected light Lr.

### [Effects and Like]

As described above, endoscope system 1 according to the present embodiment includes: insertion component 10 which includes base end 10b and tip end 10a, and is inserted into the subject at least up to tip end 10a; light source 20 which emits first light L1 and second light L2 having a hue different from a hue of first light L1; light sensor 30 which is sensitive to a wavelength of at least one of first light L1 or second light L2; and controller 40 which controls light source 20 based on the result of detection performed by light sensor 30. Insertion component 10 includes: first optical fiber 11 which guides first light L1 and second light L2 emitted from light source 20 from base end 10b to tip end 10a so that first light L1 and second light L2 are emitted from tip end 10a as illumination light L; and second optical fiber 12 which guides reflected light Lr of illumination light L emitted from tip end 10a from tip end 10a to base end 10b. Light sensor 30 receives reflected light Lr guided by second optical fiber 12. Controller 40 controls light source 20 based on the intensity of reflected light Lr received by light sensor 30 to stop the emission of the at least one of first light L1 or second light L2.

In this way, it is possible to stop the light emission performed by light source 20 based on the intensity of reflected light Lr. The intensity of reflected light Lr differs between a case where tip end 10a of insertion component 10 is located inside the subject and a case where tip end 10a of insertion component 10 is located outside the subject. Hence, it is possible to stop the light emission based on the intensity of reflected light Lr when tip end 10a is located outside the subject. Therefore, it is possible to suppress the entry of strong light into the human eye.

Reflected light Lr is guided by second optical fiber 12 to light sensor 30, and thus light sensor 30 does not need to be arranged at tip end 10a of insertion component 10. If light sensor 30 is arranged at tip end 10a, wiring cables for supplying power to light sensor 30 and for reading signals are needed. Hence, by the influences of light sensor 30 and the wiring cables, it is difficult to reduce the diameter of insertion component 10.

By contrast, in the endoscope system according to the present embodiment, it is possible to eliminate the electrical configuration from insertion component 10 (electricity-free), and thus the reduction in the diameter of insertion component 10 can be realized. The electricity-free configuration is realized, and thus insertion component 10 can be disposed of (is disposable).

For example, controller 40 compares, with the threshold value, the relative value of the intensity of reflected light Lr of the predetermined wavelength received by light sensor 30 to the intensity of reflected light Lr of the predetermined wavelength when tip end 10a is inserted into the subject. When the relative value is higher than the threshold value, controller 40 stops the emission of the at least one of first light L1 or second light L2.

In this way, the relative value of the intensity of reflected light Lr is utilized, and thus it is possible to make a determination according to the property of light source 20. Hence, it is possible to more accurately determine whether tip end 10a is located inside or outside the subject.

For example, the predetermined wavelength is the peak wavelength of second light L2. Controller 40 stops the emission of second light L2 when the relative value is higher than the threshold value.

In this way, the wavelength of the light emitted by light source 20 can be the same as the wavelength of the light received by light sensor 30, and thus it is possible to suppress the influences of external light and the like. Hence, it is possible to more accurately determine whether tip end 10a is located inside or outside the subject.

For example, light sensor 30 includes sensor 31 which is sensitive to a wavelength band including the peak wavelengths of first light L1 and second light L2.

In this way, even when variations in the light emitted by light source 20 occur, light sensor 30 can receive the light corresponding to the wavelength of the emitted light. Hence, it is possible to determine whether tip end 10a is located inside or outside the subject.

For example, endoscope system 1 may include light sensor 30B instead of light sensor 30. Light sensor 30B includes filter 32 which transmits the light of the predetermined wavelength and sensor 31 which is sensitive to the predetermined wavelength and receives the light that has passed through filter 32.

In this way, it is possible to obtain the intensity of the light of the predetermined wavelength with a simple configuration.

For example, endoscope system 1 may include light sensor 30A instead of light sensor 30. Light sensor 30A includes a plurality of sensors which are respectively sensitive to different wavelength bands. The sensors include: first sensor 31g which is sensitive to the peak wavelength of first light L1; and second sensor 31v which is sensitive to the peak wavelength of second light 31v.

In this way, the reflected light of first light L1 and the reflected light of second light L2 can be received by separate sensors, and thus it is possible to accurately obtain the intensities of the reflected light. Hence, it is possible to accurately determine whether tip end 10a is located inside or outside the subject.

For example, light source 20 includes: first light source 21 which emits third light L3; second light source 22 which emits second light L2; and wavelength converter 23 including a fluorescent substance which absorbs a part of third light L3 and emits fourth light L4 having a hue different from a hue of third light L3. Wavelength converter 23 is disposed in the optical path of third light L3, and emits, as first light L1, at least a part of combined light of the part of third light L3 and fourth light L4 when third light L3 enters wavelength converter 23.

In this way, it is possible to emit first light L1 which has a specific wavelength component and second light L2. Hence, for example, a special observation such as NBI can be performed.

For example, light source 20 further includes: filter 24 which transmits light of the specific wavelength included in the combined light, and light source 20 emits, as first light L1, the light which has passed through filter 24.

In this way, it is possible to emit first light L1 which has a specific wavelength component. Hence, for example, the special observation such as the NBI can be performed.

For example, first light source 21 is a blue laser light source, and second light source 22 is a violet laser light source.

In this way, it is possible to perform the NBI which is an example of the special observation. Since the laser light source emits narrow-band light, it is possible to accurately perform the NBI.

For example, first light L1 is green light, and second light L2 is violet light.

In this way, it is possible to perform the NBI which is an example of the special observation.

### (Others)

Although the endoscope system according to the present invention has been described above based on the embodiment described above and the like, the present invention is not limited to the embodiment described above.

For example, light source 20 does not need to include wavelength converter 23 and filter 24. Light source 20 may include, as first light source 21, a green laser light source which emits first light L1.

Light source 20 may emit blue light instead of one of green light and violet light. For example, light source 20 may omit wavelength converter 23 and filter 24, and apply, as first light L1, third light L3 emitted by first light source 21 serving as a blue laser light source to an end of first optical fiber 11 without third light L3 being processed. In this way, light source 20 can emit blue light and violet light, and thus it is possible to perform the NBI which utilizes blue light and violet light.

Alternatively, light source 20 may omit second light source 22 serving as a violet laser light source. A part of third light L3 emitted by first light source 21 may be emitted as second light L2 without being passed through wavelength converter 23 and filter 24. In this way, light source 20 can emit green light and blue light, and thus it is possible to perform the NBI which utilizes green light and violet light.

The special observation which can be performed using endoscope system 1 may be indocyanine green (ICG) fluorescence imaging. Alternatively, endoscope system 1 may be utilized for treatments such as photodynamic therapy (PDT). In these cases, as light source 20, light sources in which the peak wavelengths, half widths, and the like of first light L1 and second light L2 are adjusted according to the type of special observation and the type of PDT can be utilized.

Embodiments obtained by performing various types of variations conceived by a person skilled in the art on the embodiment and embodiments realized by arbitrarily combining the constituent elements and functions in the embodiment without departing from the spirit of the present invention are also included in the present invention.

### REFERENCE SIGNS LIST

1 endoscope system
10 insertion component
10a tip end
10b base end
11 first optical fiber (first light guide member)
12 second optical fiber (second light guide member)
20 light source
21 first light source
22 second light source
23 wavelength converter
24, 32 filter
30, 30A, 30B light sensor
31 sensor
31g first sensor
31v second sensor
40 controller
L illumination light
L1 first light
L2 second light
L3 third light
L4 fourth light
Lr reflected light

1. An endoscope system comprising:
an insertion component which includes a base end and a tip end, and is inserted into a subject at least up to the tip end;
a light source which emits first light and second light having a hue different from a hue of the first light;
a light sensor which is sensitive to a wavelength of at least one of the first light or the second light; and
a controller which controls the light source based on a result of detection performed by the light sensor,
wherein the insertion component includes:
a first light guide member which guides, from the base end to the tip end, the first light and the second light emitted from the light source so that the first light and the second light are emitted from the tip end as illumination light; and
a second light guide member which guides, from the tip end to the base end, reflected light of the illumination light emitted from the tip end,
the light sensor receives the reflected light guided by the second light guide member, and
the controller controls the light source based on an intensity of the reflected light received by the light sensor to stop emission of at least one of the first light or the second light.

2. The endoscope system according to claim 1,
wherein the controller compares, with a threshold value, a relative value of an intensity of the reflected light of a predetermined wavelength received by the light sensor to an intensity of the reflected light of the predetermined wavelength when the tip end is inserted into the subject, and when the relative value is higher than the threshold value, the controller stops the emission of the at least one of the first light or the second light.

3. The endoscope system according to claim 2,
wherein the predetermined wavelength is a peak wavelength of the second light, and
the controller stops emission of the second light when the relative value is higher than the threshold value.

4. The endoscope system according to claim 2,
wherein the light sensor includes:
a filter which transmits light of the predetermined wavelength; and
a sensor which is sensitive to the predetermined wavelength and receives light that has passed through the filter.

5. The endoscope system according to claim 1,
wherein the light sensor includes a sensor which is sensitive to a wavelength band including peak wavelengths of the first light and the second light.

6. The endoscope system according to claim 1,
wherein the light sensor includes a plurality of sensors which are respectively sensitive to different wavelength bands, and
the plurality of sensors include:
a first sensor which is sensitive to a peak wavelength of the first light; and
a second sensor which is sensitive to a peak wavelength of the second light.

7. The endoscope system according to claim 1,
wherein the light source includes:
a first light source which emits third light;
a second light source which emits the second light; and
a wavelength converter including a fluorescent substance which absorbs a part of the third light and emits fourth light having a hue different from a hue of the third light, and
the wavelength converter is disposed in an optical path of the third light, and emits, as the first light, at least a part of combined light of the part of the third light and the fourth light when the third light enters the wavelength converter.

8. The endoscope system according to claim 7,
wherein the light source further includes a filter which transmits light of a specific wavelength included in the combined light, and
the light source emits, as the first light, the light which has passed through the filter.

9. The endoscope system according to claim 7,
wherein the first light source is a blue laser light source, and
the second light source is a violet laser light source.

10. The endoscope system according to claim 1,
wherein the first light is green light, and
the second light is violet light.
